(19) United States
(12) Patent Application Publication
(10) Pub. No.: US 2022/0273228 A1
(43) Pub. Date: Sep. 1, 2022

(54) SYSTEM FOR ASSISTING IN THE SIMULATION OF THE SWALLOWING OF A PATIENT AND ASSOCIATED METHOD

(71) Applicant: SWALLIS MÉDICAL, STRASBOURG (FR)

(72) Inventor: Linda NICOLINI, TOULOUSE (FR)

(21) Appl. No.: 17/629,116

(22) PCT Filed: Jul. 7, 2020
(86) PCT No.: PCT/EP2020/069076

§ 371 Date: Jan. 21, 2022

(30) Foreign Application Priority Data
Jul. 24, 2019 (FR) FR1908396

(51) Int. Cl. A61B 5/00; A61B 7/00; G16H 50/50; G16H 20/70

(52) U.S. Cl. CPC A61B 5/4205; A61B 7/008; G16H 50/50; G16H 20/70; A61B 5/7445; A61B 2562/0219; A61B 5/6814

(57) ABSTRACT

A system includes a device for detecting the swallowing of a patient including at least one sensor for detecting swallowing configured to measure a swallowing signal, a processor for processing the swallowing signal connected to the device for detecting swallowing and configured to characterize the swallowing signal. The system includes an augmented reality or virtual reality headset configured to display virtual content to the patient, a virtual content processor connected to the processor for processing the swallowing signal and to the augmented reality or virtual reality headset, the virtual content processor being configured to deliver the virtual content to the augmented reality or virtual reality headset and to adapt the virtual content delivered according to the swallowing signal received from the processor for processing the swallowing signal.

### TECHNICAL FIELD OF THE INVENTION

The technical field of the invention is that of the detection of swallowing.

The present invention relates to a system for assisting in the simulation of the swallowing of a patient and in particular a system comprising a collar device and at least one virtual reality or augmented reality headset.

### TECHNOLOGICAL BACKGROUND OF THE INVENTION

Patients with swallowing disorders, also called “dysphagia”, present difficulties in swallowing food, for example lack of coordination in the conveyance of food items between the mouth and the stomach passing through the pharynx and the oesophagus, and risks of false passages. Concerning false passages, one speaks of penetration if the food item enters into the larynx but remains above the glottis, the vocal chords, and of aspiration if it passes the vocal chords. Aspiration leads to one of the corporal responses, normally a cough, but if a sensitivity disorder exists, the aspiration may be without cough and thus silent.

Dysphagia may for example appear in patients after a cerebrovascular accident (CVA), after a craniocerebral trauma (CCT), after amyotrophic lateral sclerosis (ALS) or in patients with Alzheimer's disease or neurodegenerative diseases.

In order to detect the dysphagia level of a patient, for example to evaluate the risk of false passage, the type, the cause and/or the level of seriousness of the dysphagia, numerous examination methods have been developed.

The most common and the most widely used, the reference for swallowing examinations, is video fluoroscopic swallowing study (VFSS). VFSS is an invasive method consisting in food stimulation by different boluses of different sizes and textures, comprising barium, coupled to imaging of the mouth, the throat and the oesophagus by X-rays. Barium is opaque to X-rays and thus makes it possible to monitor the conveyance of the bolus between the mouth and the oesophagus by radioscopy. The texture of the boluses proposed to the patient may for example be classified according to the IDDSI (International Dysphagia Diet Standardisation Initiative), which defines texture levels ranging from 0 to 7: texture levels 0 to 4 correspond to “liquid” to “thick” products that may be proposed to the patient with a syringe, and levels 4 to 7 may be proposed to the patient with a fork, chopsticks or fingers, and correspond to products ranging from “mixed” to “normal”.

However, VFSS has several drawbacks: the first being the use of X-rays and barium, the patients becoming more vulnerable to the effects of barium ionising radiation. Another drawback of VFSS is its invasive character during an examination, the patient is brought to swallow several boluses of different textures and sizes, which can be very tiring for someone with dysphagia and make him run non-negligible risks of false passages. Further, VFSS cannot be used to analyse rehabilitation exercises of the patient because the exposure time to X-rays must necessarily be very limited. The invasive character of VFSS here poses a further problem, notably because rehabilitation exercises are repetitive and the fact of having to swallow boluses of different sizes and textures comprising barium may discourage the patient in his progression and lead to difficulties in attaining satisfactory results, in addition to having a cost.

Another technique well known to those skilled in the art is endoscopy, or fibroscopy. This method, which is also invasive, consists in the insertion of an endoscope, or fibroscope, an optical tube provided with a lighting system which may be coupled to a video camera. Endoscopy, compared to VFSS, has the advantage of being able to be used at the patients bedside, without the need for a bulky fluoroscopy system. However, endoscopy remains invasive, the presence of the endoscope altering the physiology of swallowing. Further, the observation of swallowing takes place only from the upper viewpoint of the endoscope, it is thus not possible to know, with endoscopy, what happens from the moment of closing of the epiglottis.

These methods also require the expertise of professionals trained in the detection of dysphagia and the severity thereof.

Faced with the need for non-invasive methods for evaluating dysphagia, swallowing accelerometry has been developed, notably thanks to miniaturisation and improvements in the precision of electronic accelerometers. Collar devices comprising an accelerometer have been developed for the acquisition of swallowing accelerometry signals at the level of the larynx. Indeed, swallowing may be divided into three phases:
the oral phase of swallowing is voluntary and comprises the posterior movement of the tongue and the hyoid bone,
the laryngo-pharyngeal phase is automatic and reflex and comprises the laryngeal movement, the raising of the hyoid bone, the closing of the epiglottis and the passage of the bolus to the oesophageal orifice, and
the oesophageal phase is reflex and comprises peristaltic contraction, repositioning of the hyoid bone and the larynx and reopening of the epiglottis.

The capture of these signals enables their processing by computer and their characterisation with respect to these three swallowing phases, and automatic classification techniques have thus been applied to these signals in the prior art for the detection of dysphagia, aspirations, silent false passages and/or for the evaluation of dysphagia level.

Such collar devices may further comprise a laryngeal sound sensor, for example a microphone, to measure the swallowing sound, for example in order to filter and analyse breathing sounds, coughs and sounds linked to the voice of the patient. The microphone can also enable the detection of swallowing, for example by detecting the laryngeal ascension sound corresponding to the ascension of the larynx when the bolus is localised in the oropharynx and/or the hypopharynx, the sound of opening of the upper sphincter corresponding to the transit of the bolus through the upper sphincter, and the laryngeal release sound corresponding to the descent and to the opening of the larynx when the bolus has reached the oesophagus, as described by Sylvain Morinière et al. in “Origin of the Sound Components During Pharyngeal Swallowing in Normal Subjects”, Dysphagia, 2008.

These collar devices may be used for the detection and the analysis of swallowing in a patient with dysphagia, or for the analysis of swallowing rehabilitation exercises. In such cases, either the patient has to simulate swallowing, which is complex because it is difficult to place oneself in situation without really swallowing food, or the patient has to swallow boluses of different sizes and textures, which “cancels” the non-invasive advantage of collar devices, the patient still having to carry out tiring exercises of swallowing real foods and thus taking non-negligible risks of aspirations and false passages.

There thus exists a need to be able to study the dysphagia of a patient and to allow him to rehabilitate himself to swallowing without food enticement.

### SUMMARY OF THE INVENTION

The invention offers a solution to the aforementioned problems, by enabling a non-invasive study of the dysphagia of a patient and to carry out swallowing rehabilitation exercises without food enticement.

One aspect of the invention thus relates to a system comprising:
A device for detecting swallowing of a patient comprising at least one sensor for detecting swallowing configured to measure a swallowing signal,
A processor for processing the swallowing signal connected to the device for detecting swallowing, configured to characterise the swallowing signal,
   the system being characterised in that it comprises:
A virtual reality or augmented reality headset, configured to display a virtual content to the patient,
A virtual content processor connected to the processor for processing the swallowing signal and to the virtual reality or augmented reality headset, said virtual content processor being configured to deliver the virtual content to the virtual reality or augmented reality headset and to adapt the delivered virtual content as a function of the swallowing signal received from the processor for processing the swallowing signal.

Thanks to the invention, it is possible for a patient to simulate swallowing of boluses of different sizes and textures without having to actually swallow these boluses of different sizes and textures. Indeed, the laryngo-pharyngeal phase and the oesophageal phase being reflex phases, the visual stimulation of the patient realised thanks to the virtual content displayed by the virtual reality or augmented reality headsets enables these phases to be carried out without food enticement. To study his swallowing and/or to carry out swallowing rehabilitation exercises, the patient simply has to initiate swallowing by carrying out an oral phase, then the laryngo-pharyngeal and oesophageal phases are carried out automatically because they are reflex phases, which depend on the visual stimulation by the virtual content displayed by the virtual reality or augmented reality headset of the system.

Further, the system is capable of adapting the virtual content displayed as a function of the swallowing detected by the device for detecting swallowing. Indeed, an advantage of the system according to the invention is that the virtual content processor is connected to the processor for detecting swallowing. Further, the analysis of swallowing remains possible thanks to the processor for detecting swallowing, the signal for detecting swallowing thus being accessible to display on a computer or processing by a computer or by a practitioner. Thus, the system for assisting in the simulation of the swallowing of a patient according to the invention enables at a same time the analysis of the swallowing of the patient while stimulating him visually in order not to have food enticement.

Apart from the characteristics that have been set out in the preceding paragraph, the system for assisting in the simulation of the swallowing of a patient according to one aspect of the invention may have one or more complementary characteristics among the following, considered individually or according to all technically possible combinations thereof:
the sensor for detecting swallowing is a microphone for detecting a swallowing sound or an accelerometer for detecting a swallowing movement,
the device for detecting swallowing further comprises at least one sensor among heart rate, body temperature, sweating, breathing sound, respiratory rate, muscle activity (EMG) sensors.
the characterisation of the swallowing signal by the processor for processing the swallowing signal comprises a classification of the swallowing signal, in that the processor for processing the swallowing signal is further configured to send to the virtual content processor the class in which the swallowing signal has been classified and in that the adaptation of the virtual content by the virtual content processor is carried out as a function of the class received,
the virtual content comprises a food component and the swallowing signal is classified in a class corresponding to correct swallowing or in a class corresponding to incorrect swallowing and:
   if the class received by the virtual content processor is a class corresponding to correct swallowing, the virtual content processor is configured to adapt the virtual content delivered to the augmented reality or virtual reality headset by increasing the size of the food component and/or the texture level of the food component comprised in the delivered virtual content and by sending the adapted virtual content to the virtual reality or augmented reality headset;
   if the class received by the virtual content processor is a class corresponding to incorrect swallowing, the virtual content processor is configured to adapt the virtual content delivered to the augmented reality or virtual reality headset by decreasing the size of the food component and/or the texture level of the food component comprised in the delivered virtual content and by sending the adapted virtual content to the virtual reality or augmented reality headset;
if the virtual content processor is configured in a “rehabilitation” mode and if the class received by the virtual content processor is a class corresponding to incorrect swallowing, the virtual content processor is configured to not carry out the adaptation of the virtual content and to deliver the same virtual content to the virtual reality or augmented reality headset,
the virtual content processor adapts the virtual content to the swallowing signal by delivering to the virtual reality or augmented reality headset a virtual content comprising another food component of smaller size than the food component delivered previously if the swallowing signal corresponds to incorrect swallowing or if the virtual content processor receives a command to change the size of the food component,
the virtual content processor adapts the virtual content to the swallowing signal by delivering to the virtual reality or augmented reality headset a virtual content comprising another food component of size larger than the food component delivered previously if the swallowing signal corresponds to correct swallowing or if the virtual content processor receives a command to change the size of the food component,
the virtual content processor adapts the virtual content to the swallowing signal by delivering to the virtual reality or augmented reality headset a virtual content comprising another food component of texture level lower than the food component delivered previously if the swallowing signal corresponds to incorrect swallowing or if the virtual content processor receives a command to change the texture of the food component,
the virtual content processor adapts the virtual content to the swallowing signal by delivering to the virtual reality or augmented reality headset a virtual content comprising another food component of texture level higher than the food component delivered previously if the swallowing signal corresponds to correct swallowing or if the virtual content processor receives a command to change the texture of the food component.

Another aspect of the invention relates to a method for assisting in the simulation of the swallowing of a patient, the method comprising the steps of:
Sending a virtual content by a virtual content processor to a virtual reality or augmented reality headset;
Displaying the virtual content by the virtual reality or augmented reality headset;
Measuring at least one swallowing signal by a device for detecting swallowing;
Sending the swallowing signal by the device for detecting swallowing to a processor for processing the swallowing signal;
Classification of the swallowing signal by the processor for processing the swallowing signal;
Sending, by the processor for processing the swallowing signal, to the virtual content processor, the class in which the swallowing signal has been classified;
Adaptation, by the virtual content processor, of the virtual content delivered to the augmented reality or virtual reality headset as a function of the class received;

Apart from the characteristics that have been mentioned in the preceding paragraph, the method for assisting in the simulation of the swallowing of a patient according to one aspect of the invention may have one or more complementary characteristics among the following, considered individually or according to all technically possible combinations thereof.
the virtual content comprises a food component, at the classification step the swallowing signal is classified in a class corresponding to correct swallowing or in a class corresponding to incorrect swallowing and the adaptation of the virtual content by the virtual content processor comprises the following sub-steps:
   If the class received by the virtual content processor is a class corresponding to correct swallowing:
      A sub-step of increasing, by the virtual content processor, the virtual content delivered to the augmented reality or virtual reality headset by increasing the size and/or the texture level of the food component comprised in the delivered virtual content and by sending the adapted virtual content to the virtual reality or augmented reality headset,
   If the class received by the virtual content processor is a class corresponding to incorrect swallowing:
      A sub-step of decreasing, by the virtual content processor, the virtual content delivered to the augmented reality or virtual reality headset by decreasing the size and/or the texture level of the food component comprised in the delivered virtual content and by sending the adapted virtual content to the virtual reality or augmented reality headset.
if the virtual content processor is configured in a “rehabilitation” mode and if the class received by the virtual content processor is a class corresponding to incorrect swallowing, the adaptation of the virtual content is not carried out and the same virtual content is delivered to the virtual reality or augmented reality headset.

The invention and the different applications thereof will be better understood on reading the description that follows and by examining the figures that accompany it.

### BRIEF DESCRIPTION OF THE FIGURES

The figures are presented for indicative purposes and in no way limit the invention.

FIG. 1 shows a schematic representation of the system for assisting in the simulation of the swallowing of a patient according to the invention.

FIG. 2 shows a detailed schematic representation of the system for assisting in the simulation of the swallowing of a patient according to the invention.

FIG. 3 shows a schematic representation of a display of virtual content comprising a food component by a virtual reality or augmented reality headset.

FIG. 4 shows a schematic representation of the method for assisting in the simulation of swallowing of a patient according to the invention.

### DETAILED DESCRIPTION

The figures are presented for indicative purposes and in no way limit the invention.

Unless stated otherwise, a same element appearing in the different figures has a single reference.

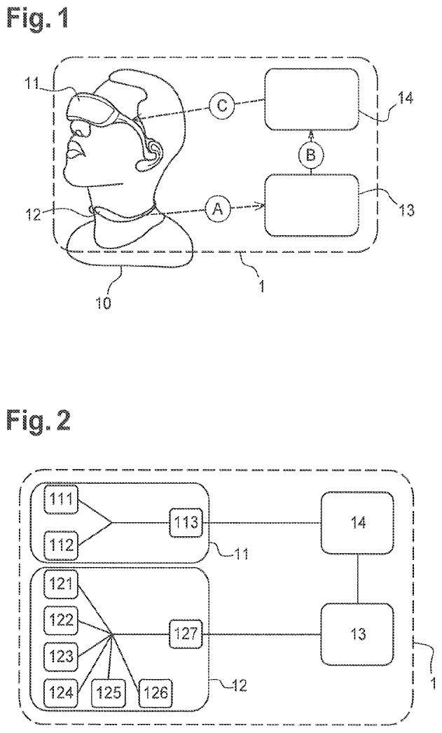

FIG. 1 shows a schematic representation of the system 1 for assisting in the simulation of swallowing of a patient according to the invention.

As represented in FIG. 1, the patient 10 is equipped with a virtual reality or augmented reality headset 11 and a device for detecting swallowing 12.

The system 1 for assisting in the simulation of swallowing of a patient according to the invention comprises the virtual reality or augmented reality headset 11, the device for detecting swallowing 12, the processor for processing the swallowing signal 13 and the virtual content processor 14.

The system 1 is “non-invasive” in that it makes it possible to carry out swallowing exercises without food enticement, that is to say without having to swallow boluses.

Virtual reality consists in immersing a user of a virtual reality headset in a virtual environment. To do so, the virtual reality headset uses stereoscopy, creating a three-dimensional environment in which the user of the virtual reality headset can move about. A virtual reality headset displays virtual content in three dimensions, in a stereoscopic manner, for example by using two screens, one for each eye of the user, as implemented by the “Oculus Rift®” or the “HTC Vive®” virtual reality headsets, or for example on a screen divided into two parts, one part for each eye of the patient 10, as proposed by the “Samsung Gear VR®” virtual reality headset. Virtual reality headsets may be associated with virtual joysticks to enable the user to interact with the virtual environment created.

Augmented reality consists in superimposing virtual elements on the real environment of a user of an augmented reality headset. To do so, the augmented reality headset takes one or more images of the real environment of the user, for example using one or more cameras situated on the augmented reality headset, to recreate digitally the real environment of the user. Next, the augmented reality headset superimposes on the images taken a virtual content in two or three dimensions with which the user of the augmented reality headset can interact. Certain augmented reality headsets display on two screens, one for each eye, the images of the real environment taken by the cameras of the headset as well as the virtual content superimposed on the real environment. The most recent augmented reality headsets, such as the “Microsoft HoloLens®” or smart glasses type headsets, only display the virtual content to superimpose on “waveguide” type displays, thus displaying the virtual content on the real environment without retransmitting the real environment on a screen. Indeed, “waveguide” type displays are transparent, the user thus being able to see the real environment through these displays. In such types of augmented reality headsets, the cameras are still present to calculate the position of the virtual content to superimpose compared to the real environment. The augmented reality headsets may be associated with joysticks to interact with the virtual content, and/or to detect movements of the arms and hands of the user to interact in a more natural manner with the virtual content.

The virtual reality or augmented reality headset 11 is a headset configured to display a virtual content to the patient 10. This virtual content may be superimposed on the real environment when the headset 11 used is an augmented reality headset, or then this virtual content may be comprised in the virtual environment created when the headset 11 used is a virtual reality headset.

FIG. 2 shows a detailed schematic representation of the system 1 for assisting in the simulation of the swallowing of a patient 10 according to the invention.

In FIG. 2, the device for detecting swallowing 12 and the virtual reality or augmented reality headset 11 of the system 1 are represented in a detailed manner.

The virtual reality or augmented reality headset 11 comprises a display device 111, an audio content streaming device 112 and a processor 113.

The display device 111 enables the display of the virtual video content to the user of the headset 11 and may comprise two screens, one for each eye, to produce a stereoscopic display. These two screens may be liquid crystal display (LCD) screens, or “waveguide” type screens as described previously. The display device 111 may comprise only one screen divided into two parts, one for each eye.

The audio content streaming device 112 enables the streaming of audio content to the user, in relation with the virtual content video displayed to the user of the headset 11 by the display device 111. The audio content streaming device 112 may comprise one or more loudspeakers, one or more headphones, or any other type of device enabling audio streaming. The virtual reality or augmented reality headset 11 may not comprise an audio content device 112.

The processor 113 of the virtual reality or augmented reality headset is configured to produce an image displayable on the display device 112, to superimpose virtual content on a real or virtual environment and to receive a virtual content and/or a command comprising an indication of a virtual content to display from the virtual content processor 14. To do so, the virtual reality or augmented reality headset 11 via its processor 113 and the virtual content processor 14 are connected. This interfacing may be wired or wireless.

The device for detecting swallowing 12 of the system 1 according to the invention comprises at least one sensor for detecting swallowing 121 configured to measure a swallowing signal of the patient 10. This sensor for detecting swallowing 121 may for example be an accelerometer situated at the level of the larynx. It can then measure a swallowing signal of the patient 10, for example a signal of laryngeal movement corresponding to swallowing or any other movement making it possible to characterise swallowing. The sensor for detecting swallowing 121 may for example be a microphone, the swallowing signal of the patient 10 measured then being a laryngeal sound, or any other sound making it possible to characterise swallowing. The sensor for detecting swallowing 121 may be any sensor capable of measuring a swallowing signal making it possible to characterise swallowing of the patient 10. Further, the device for detecting swallowing 12 may comprise a plurality of sensors for detecting swallowing 121, for example a combination of a microphone and an accelerometer in order to improve the precision and the reliability of swallowing detection.

The device for detecting swallowing 12 may for example be a collar device for detecting swallowing, as represented in FIG. 1 and such as well known to those skilled in the art.

Further, the device for detecting swallowing 12 may comprise at least one sensor among a heart rate sensor 122, a body temperature sensor 123, a sweating sensor 124, a breathing sound sensor 125, a respiratory rate sensor 126, a muscular activity sensor (not represented). The device for detecting swallowing 12 represented in FIG. 2 may comprise all the aforementioned sensors, but the invention 10 may also only comprise one of the aforementioned sensors or any possible combination of the aforementioned sensors. The sensors 122 to 126 make it possible to know precisely the state of the patient 10 during a swallowing rehabilitation exercise or during an examination of the swallowing of the patient 10. They further make it possible to realise a better adaptation of the virtual content displayed by the virtual reality or augmented reality headset 11, as will be explained hereafter in the description.

The device for detecting swallowing 12 further comprises a processor 127, configured to receive data coming from the sensors 121 to 126 and to transmit said data to the processor for processing the swallowing signal 13 with which it is interfaced.

The processor for processing the swallowing signal 13 is configured to process the swallowing signal, that is to say a data exchange A represented in FIG. 1, to the virtual content processor 14 with which the processor for processing the swallowing signal 127 is interfaced. The swallowing signal sent by the device for detecting swallowing 12 and received by the processor for processing the swallowing signal 13 during the data exchange A may comprise only one signal characterising swallowing, coming from the sensor for detecting swallowing 121. This signal may also comprise several other signals coming from the sensors 122 to 126. The processing of the swallowing signal comprises the classification of the swallowing signal, for example in a class corresponding to correct swallowing or in a class corresponding to incorrect swallowing. This classification may be carried out in a manner known to those skilled in the art by an automatic learning algorithm using a statistical model or instead a neural network. The signals coming from the sensors 122 to 126 can enable the classification model to be more precise and more reliable in its decision taking to characterise a swallowing signal as being “incorrect”, that is to say representative of dysphagia of the studied patient 10, for example a swallowing presenting a risk of false passage or aspiration, or “correct”, that is to say representative of swallowing not characteristic of dysphagia of the studied patient 10. The processor for processing the swallowing signal 13 may further be interfaced with a computer, a database, or another swallowing signal processing device for future analysis of the temporal evolution of the swallowing of the patient 10, for presentation to the patient for example in order to create biofeedback, or for any other use of the swallowing signal.

Once classified, the signal received from the device for detecting swallowing 12 by the processor for processing the swallowing signal 13 is sent, with its classification, by the processor for processing the swallowing signal 13 to the virtual content processor 14 in a data exchange B represented in FIG. 1. It is also possible to send the classification of the swallowing signal only rather than the swallowing signal and the classification of the swallowing signal, in order to minimise the exchanges of data between the processor for processing the swallowing signal 13 and the virtual content processor 14.

The virtual content processor 14 connected to the processor for processing the swallowing signal 13 and to the virtual reality or augmented reality headset 11 is configured to deliver a virtual content to the virtual reality or augmented reality headset 11 and to adapt the delivered virtual content as a function of the swallowing signals received from the processor for processing the swallowing signal 13. Thus, in the data exchange B represented in FIG. 1, the virtual content processor 14 receives the swallowing signal as well as its classification carried out by the processor for processing the swallowing signal 13, or its classification only. The adaptation of the virtual content delivered by the virtual content processor 14 to the virtual reality or augmented reality headset 11 makes it possible to evaluate the evolution of the patient 10 for whom the swallowing is examined and/or to propose to him rehabilitation exercises without the presence of a practitioner.

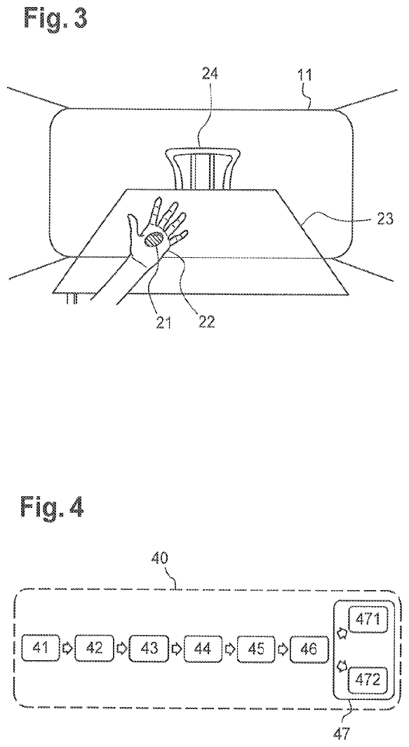

FIG. 3 shows a schematic representation of a display of virtual content 21 comprising a food component by a virtual reality or augmented reality headset 11.

In FIG. 3 is represented an environment of the user 10 of the virtual reality or augmented reality headset 11. This environment comprises a table 23, a chair 24 and a virtual content 21. When the headset 11 is a virtual reality headset, the environment of the user 10 is then a virtual environment. Thus, the table 23 and the chair 24 are virtual objects. When the headset 11 is an augmented reality or mixed headset, the environment of the user 10 is then a real environment. Thus, the table 23 and/or the chair 24 may be real objects. At least one of these two components of the environment of the user may be virtual. The user 10 has, in FIG. 3, a view of his hand 22. This hand 22 may be a real image of his own hand, when the headset 11 is an augmented reality headset, or it may be a virtual object representing his hand when the headset 11 is a virtual reality headset.

As represented in FIG. 3, a virtual content 21 comprising a food component is displayed to the user 10 of the virtual reality or augmented reality headset 11. As explained in the preceding paragraph, this virtual content may be inscribed in a virtual environment or in a real environment. For an impression of realism, it could be possible to favour augmented reality and thus the real environment, the patient 10 thus having the impression of holding a food item in his hand 22. For a patient 10 presenting stress to clinical examinations for example, it could be possible to favour virtual reality and thus a virtual environment, the patient 10 being put in condition in a reassuring environment, this virtual environment being able for example to have no link with the medical field. In the case of virtual reality, the patient 10 may for example have a joystick or a remote control and the virtual reality or augmented reality headset 11 then displays a virtual representation of his arm and his hand 22.

The virtual content 21 is delivered to the virtual reality or augmented reality headset 11 by the virtual content processor 14 in a data exchange C represented in FIG. 1. The virtual content processor 14 delivers an initial virtual content to the virtual reality or augmented reality headset 11 comprising a food component 21 of determined size and texture. For example, this initial food component 21 may be a food component of smallest size and of lowest texture level to begin a dysphagia examination or a rehabilitation exercise. It is also possible to record, for example in a database, the size and the texture level at which the patient 10 had stopped at the preceding session, to recover this information at the following session in order to propose to the patient a virtual food component 21 of the texture and size at which he had stopped at the preceding session. To do so, the virtual content processor 14 may be connected to a database, either in a local manner, or through a communication network. At each new food component 21 delivered to the virtual reality or augmented reality headset 11 by the virtual content processor 14, the virtual content processor 14 can also record in the database to which he has access the texture and the size of the delivered food component 21, to retain a trace thereof and see the progression of the patient 10 in his rehabilitation exercises and/or in the dysphagia examination. These proposed rehabilitation exercises evolve according to the signals detected. For example, a subject may be animated and may jump or accelerate according to the intensity of the signals detected, thus enabling biofeedback to the patient.

On reception of the classification of the swallowing signal only or the classification and the swallowing signal, the virtual content processor 14, knowing the size and the texture of the virtual food component delivered previously, can then adapt the virtual content delivered to the virtual reality or augmented reality headset 11 on the basis of the classification of the swallowing signal received. For example, if the virtual content processor 14 receives a classification of the swallowing signal corresponding to “correct” swallowing, then the virtual content processor 14 adapts the virtual content 21 delivered to the virtual reality or augmented reality headset 11, for example by increasing the size and/or the texture level of the food component 21. It then delivers a new virtual content comprising a suitable food component 21, so that the system 1 determines the swallowing response of the patient 10 to this new food component 21, more difficult to swallow. If the virtual content processor 14 receives a classification of the swallowing signal corresponding to “incorrect” swallowing, then the virtual content processor 14 adapts the virtual content 21 delivered to the virtual reality or augmented reality headset 11, for example by decreasing the size and/or the texture level of the food component 21, or by re-proposing the same food component 21 to analyse if the swallowing response to the preceding proposition was a one-off error. To know if it is necessary to decrease the size and/or the texture level or re-propose the same food component 21, the virtual content processor 14 may comprise modes, for example an “examination” mode corresponding to the examination of swallowing, in which the size and/or the texture level are decreased, and a “rehabilitation” mode, in which the same virtual food component 21 is re-proposed to the patient 10 until said patient successfully manages correct swallowing of this virtual food component 21. Thus, it is possible to examine the dysphagia level of the patient 10 automatically, the “threshold” size and texture of the food component 21 beyond which the patient mainly realises incorrect swallowing corresponding to a determined dysphagia level, in the “examination” mode. It is also possible to analyse the evolution and the progression of the patient 10 in his rehabilitation exercises, in “rehabilitation mode”. The mode of the virtual content processor 14 may be modified by the reception of a change of mode command, sent for example by the practitioner or the patient 10 himself, for example via a computer or any other electronic device connected to the virtual content processor 14.

Further, the virtual content processor 14 can adapt the virtual content 20 that it delivers to the virtual reality or augmented reality headset 11 on reception of a command to adapt the virtual content. This command may for example be received via a communication network to which the virtual content processor is connected. A practitioner or the patient 10 himself may be the originator of this command, for example by sending it from a computer or any other electronic device connected to the communication network or directly connected to the virtual content processor 14. This command may contain an indication on the size of the virtual food component 21 to deliver to the virtual reality or augmented reality headset 11, on its texture level, on a combination of the size and the texture level of the virtual food component 21 or on the type of virtual food component 21. This indication may be a precise value of the size or texture level of the virtual food component 21 to deliver, or an indication of the size or of texture level that is larger, smaller, or equal to the size and/or to the texture of the virtual food component 21 delivered previously.

The food component 21 proposed to the patient 10 being virtual, the patient 10 is not tired physically by the swallowing examination and/or the rehabilitation exercises that he carries out, notably by using the fact that certain swallowing phases are reflex phases, that it is not possible to control for the patient 10, and which are triggered following the oral phase of swallowing, which is a voluntary phase. Thus, when the patient 10 puts to his mouth the virtual food component 21 of determined texture and size, and that he can see, he carries out the oral phase voluntarily and the following swallowing phases in a reflex manner. Thus, this allows the patient better simulation of swallowing without having to swallow multiple boluses of different sizes and textures and makes it possible to lower the costs of such exercises. Further, this allows the patient 10 to reduce the impact of the stress linked to these exercises on the result of these exercises, notably by putting him in favourable conditions thanks to a virtual environment and to the absence of real foods.

The virtual content processor 14 can further deliver a virtual content comprising several food components 21 to the virtual reality or augmented reality headset 11, in order to leave the choice to the patient 10 of the food component(s) 21 that he wishes to swallow.

In another embodiment, the virtual content displayed to the patient may not comprise any food component 21 but may entice the patient to carry out manoeuvres or to adopt positions that facilitate swallowing. These manoeuvres or positions may for example be of “effortful swallow”, “chin tuck” or “supraglottic swallow” type known to those skilled in the art. These manoeuvres may be adapted as a function of the signals received, for example by modifying the technique to perform or by proposing another technique to perform if the preceding technique has indeed been carried out.

In an alternative embodiment, the virtual content displayed to the patient 10 by the virtual reality or augmented reality headset 11 is a video game. Thus, the system 1 according to the invention may use the swallowing signals, notably the reflex phases of swallowing, to adapt the content of the video game as a function of the measured swallowing signals. For example, during frequent swallowings measured by the device for detecting swallowing 12, the processor for processing the swallowing signal 13 can classify these swallowing signals in a “stress” or “serene” class and transmit this classification as well as the swallowing signals to the virtual content processor 14, that is going to adapt the content of the video game 5 to the state of the user of the virtual reality or augmented reality headset 11 and the device for detecting swallowing 12. For example, during the detection of a state of stress of the player thanks notably to the swallowing signals, the virtual content processor 14 can adapt the game by proposing a more distressing or less distressing content as a function of the desired effect on the player. The virtual content of the video game 10 delivered by the virtual content processor 14 may comprise a food component 21.

The system 1 according to the invention may also be used for diet-linked disorders. For example, the system 1 may display different types of food components 21 to the patient 10 and analyse their attractiveness by analysing the swallowing of the patient 10 on visualising these virtual food components 21 thanks to the device for detecting swallowing 12. When an attractiveness is detected for a certain type of food that the patient 10 no longer wishes to consume or which he must no longer consume, the virtual content processor 10 can adapt the virtual content delivered to the virtual reality or augmented reality headset 11 in order to propose a negative experience in relation with this food component 21 and thus decrease its attractiveness.

FIG. 4 shows a schematic representation of the method 40 for assisting in the simulation of the swallowing of a patient 10 according to the invention.

The method 40 for assisting in the simulation of the swallowing of a patient 10 according to the invention is implemented by the system 1 according to the invention and comprises a first step 41 of sending a virtual content 21 by the virtual content processor to a virtual reality or augmented reality headset 11 in a data exchange C represented in FIG. 1. In the second step 42, the virtual content 21 is displayed by the virtual reality or augmented reality headset 11, as represented in FIG. 3. The third step 43 is a step of measuring at least one swallowing signal by the device for detecting swallowing 12, followed by a step 44 of sending the swallowing signal by the device for detecting swallowing 12 to the processor for processing the swallowing signal 13 in a data exchange A represented in FIG. 1. A step 45 of classification of the swallowing signal is next carried out. This step is carried out by the processor for processing the swallowing signal 13 and the swallowing signal may be classified in a class corresponding to correct swallowing or in a class corresponding to incorrect swallowing, or in a class corresponding to a state of the patient or in another class corresponding to another state of the patient. The swallowing signal may be classified in all types of classes making it possible to characterise the swallowing signal, as a function of the use (therapeutic, video-playful, etc.) of the invention. The method 40 further comprises a step 46 of sending, by the swallowing signal processor 13, to the virtual content processor 14, the class in which the swallowing signal has been classified, in a data exchange B represented in FIG. 1. The method 40 comprises a step 47 of adaptation, by the virtual content processor 14, of the virtual content 21 delivered to the augmented reality or virtual reality headset 11 as a function of the class received. This adaptation has been described previously in the description. For a use of examining swallowing or rehabilitation exercises, at the classification step 45, the swallowing signal is classified in a class corresponding to correct swallowing or in a class corresponding to incorrect swallowing and the step 47 of adaptation of the virtual content by the virtual content processor 14 comprises the following sub-steps:
if the class received by the virtual content processor 14 is a class corresponding to correct swallowing:
   a sub-step 471 of increasing, by the virtual content processor, the virtual content 21 delivered to the augmented reality or virtual reality headset by increasing the size and/or the texture level of the food component comprised in the delivered virtual content 21 and by sending the augmented virtual content 21 to the virtual reality or augmented reality headset 11,
If the class received by the virtual content processor 14 is a class corresponding to incorrect swallowing:
   A sub-step 472 of decreasing, by the virtual content processor 14, the virtual content 21 delivered to the augmented reality or virtual reality headset by decreasing the size and/or the texture level of the food component comprised in the delivered virtual content and by sending the adapted content to the virtual reality or augmented reality headset,

Further, the step 47 of adaptation of the virtual content of the method 40 may not be carried out if the virtual content processor 14 is configured in a “rehabilitation” mode and if the class received by the virtual content processor 14 is a class corresponding to incorrect swallowing, the same virtual content 21 then being delivered to the virtual reality or augmented reality headset 11.

1. A system comprising:
a device for detecting a swallowing of a patient comprising at least one sensor for detecting swallowing configured to measure a swallowing signal,
a processor for processing the swallowing signal connected to the device for detecting swallowing, configured to characterise the swallowing signal,
a virtual reality or augmented reality headset, configured to display virtual content to the patient,
a virtual content processor connected to the processor for processing the swallowing signal and to the virtual reality or augmented reality headset, said virtual content processor being configured to deliver the virtual content to the virtual reality or augmented reality headset and to adapt the delivered virtual content as a function of the swallowing signal received from the processor for processing the swallowing signal.

2. The system according to claim 1, wherein the sensor for detecting swallowing is a microphone for detecting a swallowing sound or an accelerometer for detecting a swallowing movement.

3. The system according to claim 1, wherein the device for detecting swallowing further comprises at least one sensor among heart rate, body temperature, sweating, breathing sound respiratory rate, muscular activity sensors.

4. The system according to claim 1, wherein the characterisation of the swallowing signal by the processor for processing the swallowing signal comprises a classification of the swallowing signal, wherein the processor for processing the swallowing signal is further configured to send to the virtual content processor the class in which the swallowing signal has been classified and wherein the adaptation of the virtual content by the virtual content processor is realised as a function of the class received.

5. The system according to claim 1, wherein the virtual content comprises a food component and wherein the swallowing signal is classified in a class corresponding to correct swallowing or in a class corresponding to incorrect swallowing and wherein:
if the class received by the virtual content processor is a class corresponding to correct swallowing, the virtual content processor is configured to adapt the virtual content delivered to the augmented reality or virtual reality headset by increasing a size and/or a texture level of the food component comprised in the delivered virtual content and by sending the adapted virtual content to the virtual reality or augmented reality headset;
if the class received by the virtual content processor is a class corresponding to incorrect swallowing, the virtual content processor is configured to adapt the virtual content delivered to the augmented reality or virtual reality headset by decreasing the size and/or the texture level of the food component comprised in the delivered virtual content and by sending the adapted virtual content to the virtual reality or augmented reality headset;

6. The system according to claim 1, wherein, if the virtual content processor is configured in a rehabilitation mode and if the class received by the virtual content processor is a class corresponding to incorrect swallowing, the virtual content processor is configured to not carry out the adaptation of the virtual content and to deliver the same virtual content to the virtual reality or augmented reality headset.

7. A method for assisting in the simulation of a swallowing of a patient, the method comprising:
sending a virtual content by a virtual content processor to a virtual reality or augmented reality headset;
displaying the virtual content by the virtual reality or augmented reality headset;
measuring at least one swallowing signal by a device for detecting swallowing;
sending the swallowing signal by the device for detecting swallowing to a processor for processing the swallowing signal;
classifying the swallowing signal by the processor for processing the swallowing signal;
sending, by the processor for processing the swallowing signal, to the virtual content processor, the class in which the swallowing signal has been classified;
adapting, by the virtual content processors, of the virtual content delivered to the augmented reality or virtual reality headset as a function of the class received.

8. The method for assisting in the simulation of the swallowing of a patient according to claim 7, wherein the virtual content comprises a food component, wherein, at the classification step, the swallowing signal is classified in a class corresponding to correct swallowing or in a class corresponding to incorrect swallowing and wherein the adaptation of the virtual content by the virtual content processor comprises the following sub-steps:
if the class received by the virtual content processor is a class corresponding to correct swallowing:
a sub-step of increasing, by the virtual content processor, the virtual content delivered to the augmented reality or virtual reality headset by increasing a size and/or a texture level of the food component comprised in the delivered virtual content and by sending the adapted virtual content to the virtual reality or augmented reality headset,
if the class received by the virtual content processor is a class corresponding to incorrect swallowing:
a sub-step of decreasing, by the virtual content processor, the virtual content delivered to the augmented reality or virtual reality headset by decreasing the size and/or the texture level of the food component comprised in the delivered virtual content and by sending the adapted virtual content to the virtual reality or augmented reality headset.

9. The method for assisting in the simulation of the swallowing of a patient according to claim 8, wherein, if the virtual content processor is configured in a rehabilitation mode and if the class received by the virtual content processor is a class corresponding to incorrect swallowing, the adaptation of the virtual content is not carried out and the same virtual content is delivered to the virtual reality or augmented reality headset.
